(19) United States
(12) Patent Application Publication
(10) Pub. No.: US 2007/0140967 A1
(43) Pub. Date: Jun. 21, 2007

(54) Agents and methods for analyzing protein interactions

(76) Inventors: Nichole Lea Wood, Niskayuna, NY (US); Mohan Mark Amaratunga, Clifton Park, NY (US); Hans Grade, Schenectady, NY (US); Faisal Ahmed Syud, Clifton Park, NY (US); Anup Sood, Clifton Park, NY (US); Reginald Donovan Smith, Rensselaer, NY (US); Ayse Betul Dinc, Troy, NY (US); Amy Casey Williams, Clifton Park, NY (US); Anthony (Tony) John Murray, Lebanon, NJ (US); Gregory Daryll Goddard, Ballston Spa, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/305,970

(22) Filed: Dec. 19, 2005

(51) Int. Cl. A61K 51/00

(52) U.S. Cl. 424/1.69

(57) ABSTRACT

Agents and methods for qualitative and quantitative analysis a protein complex or protein complexes using isotope-labeled symmetrical bifunctional crosslinkers and mass spectrometry are provided. Targeting moieties, cell permeability moieties, or affinity moieties, may be appended to the bifunctional crosslinkers. The isotope-labeled symmetrical bifunctional crosslinkers may be used in a kit or as a library.

### FIELD OF THE INVENTION

The present invention is directed to the field of protein analysis. In particular, the present invention is directed to compositions and methods for qualitative and quantitative analysis of protein-protein interactions.

### BACKGROUND

Protein-protein interactions play a central role in biological processes. The study of these interactions is an essential requirement to understanding biological processes. Some approaches for analyzing protein-protein interactions require relatively large quantities of purified protein. Other approaches only measure binary interactions (i.e., the interactions of only two proteins at a time) and have been shown to have a high rate of false positives.

Mass spectrometry (MS) is well suited to the study of proteins and protein complexes because it is highly sensitive and requires a relatively small sample quantity. Various strategies, such as co-immunoprecipitation and tandem-affinity purification, have been coupled with mass spectrometry for identifying protein complexes. These combined approaches are limited in their ability to trap weak or transient protein interactions and can only be used for complexes that remained intact following cell lysis.

Crosslinkers to capture protein-protein interactions followed by MS analysis have been used to identify protein-protein interactions, however, no methods are currently available to quantify protein-protein interactions or to compare the amount of protein-protein interactions between samples. Absolute quantification or at the least relative quantification between samples is essential for drug discovery and diagnostic applications.

Needs remain for analytical tools and methods to qualitatively and quantitatively measure and compare relative amounts of protein interactions in different samples. Accordingly, provided herein are agents and analytical methods that employ isotope-coded bifunctional crosslinkers and MS to analyze relative protein interactions between two or more samples.

### SUMMARY

The present invention disclosed herein will be made more apparent from the description, drawings, and claims that follow.

In one aspect, the present invention provides a symmetrical bifunctional cross-linking agent, comprising a pair of reactive terminal moieties positioned at opposite ends of a cleavable, isotopically labeled (e.g., C¹², C¹³; N¹⁴, N¹⁵, S³², S³⁴, O¹⁶, O¹⁷, O¹⁸; Br⁷⁹, Br⁸¹; Cl³⁵, and Cl³⁷) internal linker portion.

In some embodiments, each of the reactive terminal moieties comprises matching reactive groups selected from N-hydroxysuccinimide ester, aldehyde, acid, imidoester, aryl azide, difluorobenzene, aryl halide, carbodimide, haloacetyls, iodoacetyl groups, pyridyl disulfides, hydrazides, isocyanate, or maleimide.

In some embodiments, the internal linker portion comprises one or more cleavage sequences capable of specific or nonspecific cleavage by a chemical cleavage agent or an enzymatic cleavage agent. In some specific embodiments, the internal linker portion comprises an even number of paired matching cleavage sites, each paired cleavage site being positioned along the internal linker portion equidistant from the nearer terminal reactive moiety. In alternative embodiments, the internal linker portion comprises an odd number of cleavage sites, wherein one of the cleavage sites is positioned at or near the internal axis of the internal linker portion and the remaining cleavage site comprise paired matching cleavage sites, each paired cleavage site being positioned along the internal linker portion equidistant from the nearer terminal reactive moiety.

In some embodiments, the bifunctional crosslinker has a molecular weight between about 100 Da to about 5000 Da. In alternative embodiments, molecular weight of the bifunctional crosslinker is less than about 1000 Da.

In some further embodiments bifunctional crosslinker comprises an affinity tag, which may be an amino acid-based sequence or a nucleic-acid-based sequence (e.g., DNA, RNA, or PNA), or a small molecule (e.g., biotin).

The bifunctional crosslinker may, in some embodiments, further comprise a targeting moiety, which may comprise amino acid residues, nucleic acid residues (e.g., DNA, RNA, or PNA), or a small molecule.

In another aspect, the present invention provides methods for comparatively analyzing protein-protein interactions between proteins present in two samples including a first sample and a second sample comprising the steps of: (a) cross-linking the proteins in the first sample with the bifunctional cross-linking agent of claim 1, (b) cross-linking proteins in the second sample with an isotopic variant of the bifunctional cross-linking agent of step (a); (c) combining the first sample and second sample to produce a mixed sample; and analyzing the mixed sample. The analyzing step may employ a variety of MS techniques including MALDI-TOF, ESI-MS, LC-ESI-MS, MALDI-TOF/TOF, ESI-MS-MS, FAB, or FTICR-MS, which may be employed singly or in combination.

The disclosed methods may, further comprise an enriching step, an enzymatic or chemically cleavage step before the analyzing step. In some embodiments, the cleavage step employs a proteolytic agent. In some embodiments, the bifunctional crosslinker further comprises an affinity tag and the enriching step may comprise capturing the crosslinked protein through the affinity tag in a chromatographic matrix or other separation medium. In another embodiment, the target protein naturally expresses or is engineered to express an affinity tag and the enriching step comprises capturing the crosslinked protein through the affinity tag present on the target protein or the bifunctional cross-linker in a chromatographic matrix or other separation medium.

In some embodiments, the analyzing step may comprise determining the relative amounts of the bifunctional cross-linked protein from the first sample and the mass-shifting variant proteins from the second sample. In some embodiments, the enriching steps and the analyzing step occur in series without user intervention.

The methods provided herein may be used to analyze samples from a variety of sources. In one embodiment, a first sample and a second sample, derived from different sources, are analyzed. In some embodiments, the first sample and the second sample are derived from a single source, wherein the first sample comprises material that has been contacted with an effector agent and the second sample has not been contacted with the effector agent. In some embodiments, the first sample and second sample are derived from a mammalian subject before and after administering an effector agent to the mammalian subject.

In yet another aspect, the present invention provides differential isotopic labeling kits, comprising a pair of matching bifunctional cleavable crosslinkers, comprising a first bifunctional crosslinker and a second bifunctional crosslinker, wherein the first and the second bifunctional crosslinker have a mass shift differential greater than or equal to 2 Da. In other embodiments, the mass shift differential is greater than 4 Da. The isotopic labeling kits, may further comprise a set of matching bifunctional cleavable crosslinkers, comprising a first bifunctional crosslinker and a second bifunctional crosslinker, a third bifunctional crosslinker and so on wherein all bifunctional crosslinkers have a mass shift differential greater than or equal to 2 Da compared to the next lower mass bifunctional crosslinker. In other embodiments, the mass shift differential is greater than or equal to 4 Da.

In some embodiments, the bifunctional crosslinkers in the kit display a mass shift differential greater than or equal to 2 Da or greater than or equal to 4 Da between sequential members of the kit.

### BRIEF DESCRIPTION OF THE DRAWINGS

FIGS. 1A, 1B, and 1C depict representative chemical structures of the disclosed bifunctional including various appended moieties. In all figures X and Y represent an affinity tag, a targeting moiety or a cell permealization-enhancing moiety.

FIG. 2 depicts one workflow scheme that may be employed to practice the disclosed methods using any pair of bifunctional crosslinkers that are identical in structure but different in mass.

FIG. 3 shows a representative chemical structure of a symmetrical bifunctional crosslinkers in the form of a disucciylcystamine-bis NHS ester.

FIG. 4 shows a bifunctional crosslinkers in the form of a disuccinlycystamine-¹³C₈-bis NHS ester.

FIG. 5 depicts an optional enriching step where a crosslinker-protein fragment is isolated from an SDS-PAGE gel. Lane 1 shows melittin, lane 2 shows calmodulin, and lanes 4-9 show cross-linked calmodulin/melittin (10 μM each) after 90 minutes incubation with 700x cross-linking reagent, disuccinlycystamine-bis NHS ester.

FIG. 6: shows the MALDI-MS characterization of the bifunctional cross-linked calmodulin and melittin. Following incubation of the CaM/Melititin complex with the cross-linking reagent, disuccinlycystamine-bis NHS ester, new peaks were observed by MS corresponding to the crosslinker modified calmodulin (i.e. one-end of the is hydrolyzed) and intermolecularly cross-linked calmodulin/melittin.

FIG. 7 depicts a workflow scheme for analyzing protein interactions subsequent to the bifunctional crosslinker binding step, including optional cleavage steps (specifically, chemically mediated cleavage and enzymatically-mediated cleavage) and optional enrichment steps (gel purification, peptide extraction, and chromatographic separation), followed by MS analysis.

FIG. 8 shows the MALDI-MS identification of a modified peptide. Following the workflow described in FIG. 10, a peak was observed at M+H+=1971 Da, corresponding to calmodulin peptide (92-107) with the reduced and alkylated fragment of the cleaved bifunctional, disuccinlycystamine-bis NHS ester.

FIG. 9A and FIG. 9B show the MALDI-MS analysis of a mixed sample of isotope labeled and unlabeled modified peptides. Using a pair of bifunctional crosslinkers, disuccinlycystamine-¹³C₈-bis NHS ester and disuccinlycystamine-bis NHS ester, peaks at 1971 Da and 1975 Da (monoisotopic) were identified, corresponding to the calmodulin peptide (92-107) modified with either the isotope labeled (1975 Da) or unlabeled (1971 Da) bifunctional crosslinkers (previously cleaved, reduced, and alkylated). FIG. 9B is an expanded view of this region of the MS spectra, highlighting the isotopic pattern for both the isotope labeled and unlabeled crosslinker modified peptide.

FIG. 10 shows the MALDI-MS analysis of a 2:1 ratio of isotope labeled and unlabeled modified peptide, using a pair of bifunctional s, disuccinlycystamine-¹³C₈-bis NHS ester and disuccinlycystamine-bis NHS ester. The isotopic pattern is shown for the un-labeled (m/z=1970.9 Da, monoisotopic) and isotope labeled (m/z=1974.9 Da, monoisotopic) crosslinker modified calmodulin peptide (92-107).

FIG. 11 shows a linear regression analysis of the integrated peak areas measured from known molar ratios of isotope labeled and unlabeled crosslinker modified peptide. Peak areas were adjusted for the normal isotope abundance, yielding a calibration curve that can be used to measure molar ratios for unknown samples over the range of the regression analysis.

FIG. 12 shows a plot of the predicted molar ratios for a mixture of isotope labeled and unlabeled modified peptide. Using the method of Isotope Dilution, these ratios were calculated from separate measurements of the integrated peak areas corresponding to pure isotope labeled and unlabeled peptide.

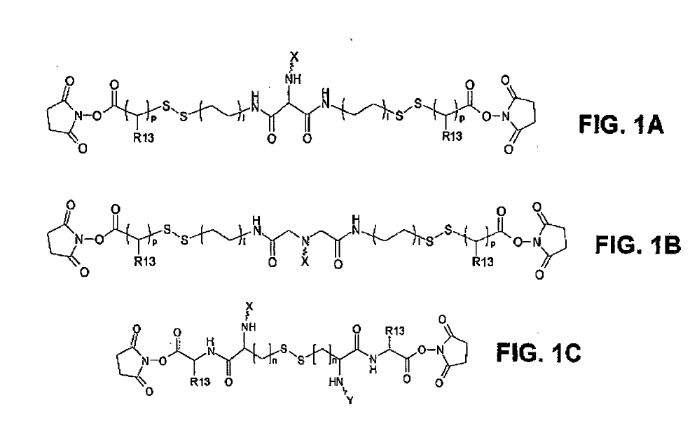

### DETAILED DESCRIPTION

Provided herein are agents and methods for qualitative and quantitative analysis a protein complex or protein complexes using isotope-coded bifunctional crosslinkers and mass spectrometry.

Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

In one series of embodiments, agents for analyzing protein interactions are disclosed. These agents comprise substantially symmetrical, cleavable isotopic labeled bifunctional crosslinkers. The disclosed bifunctional crosslinkers comprise a pair of matching reactive terminal portions positioned at opposite ends of a cleavable internal linker portion. The internal linker portion includes one or more cleavage sites, which may be cleaved by chemical cleavage agents, enzymatic cleavage agents or both chemical cleavage agents and enzymatic cleavage agents. The basic structure of the bifunctional crosslinkers may be schematically represented as:
D1---------R7----------D2

Where D1 and D2 are reactive functional moieties independently selected from reactive esters, aryl azides and other photoreactive groups (e.g. psoralans, coumarins), haloacyl, carboxyl, disulfides, maleimides, hydrazides, aldehydes, glyoxals, imidoesters where D1 and D2 are the same.

R7 is a symmetrical organic moiety of sufficient length to incorporate one or more cleavable linkages into the internal linker portion and to allow incorporation of enough isotope labels (e.g. 13C, 15N, 17O, or 18O.) to have a 2 Dalton higher mass per protein or protein fragment after cleavage of the crosslinker than what would be expected if the lighter mass isotopes were used such that the cleavage of cleavable linkages predominantly yields structurally similar crosslinker fragments on each protein or protein fragment.

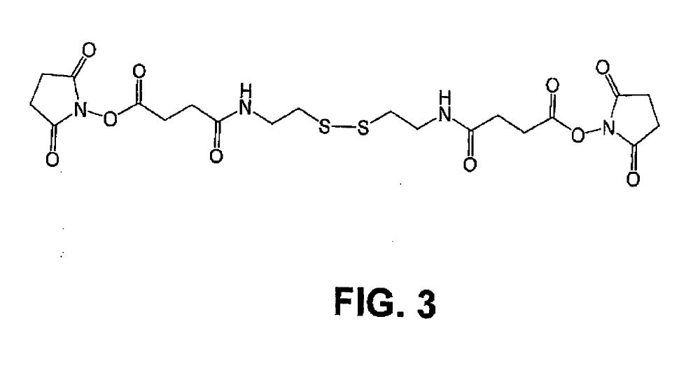

The bifunctional crosslinker may optionally include one or more appended functional moieties. Thus, the bifunctional crosslinker may include an optional targeting moiety that directs the bifunctional crosslinker to a specific protein sequence or protein fragment or adjacent portions of two or more proteins in a protein-protein complex. The bifunctional crosslinker may also include an optional affinity tag that may be used to enrich (e.g., through a chromatographic method) crosslinker modified proteins from unmodified proteins and other cellular components. Furthermore, the bifunctional crosslinker may optionally include a cell permeability-enhancing moiety, which facilitates entry of a bifunctional crosslinker into a whole cell, whether the mode of entry is passive transport or active transport. An exemplary structure is shown in FIG. 3.

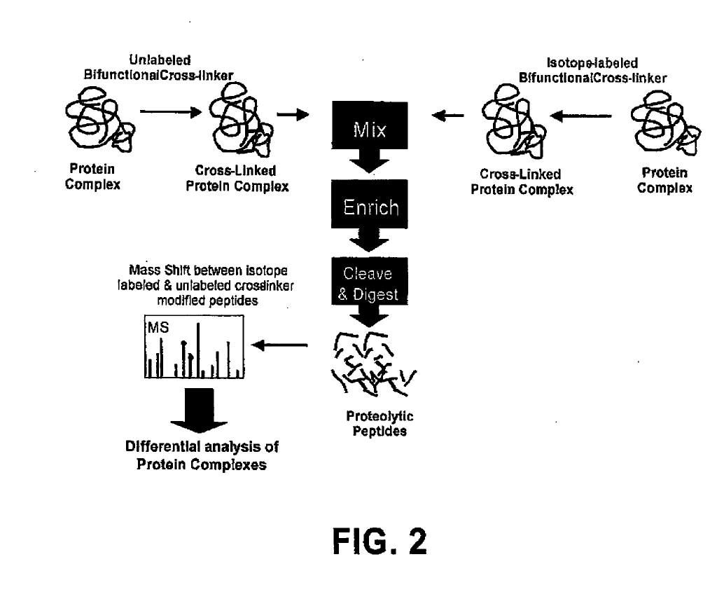

In one series of embodiments, the methods employed are schematically depicted in FIG. 2. The methods disclosed herein facilitate the identification of protein-protein interactions, and further enable the relative quantification of the protein complex abundance in various samples. Furthermore, methods employing cell permeable cross-linking agents, may be used either in vitro or in situ to analyze samples derived from a variety of samples including samples derived from tissue, serum, or cultured cells.

Definitions

To more clearly and concisely describe and point out the subject matter of the claimed invention, the following definitions are provided for specific terms, which are used in the following description and the appended claims.

As used herein the term “activatable” refers to the ability of a chemical species to transition from non-reactive state to a reactive state by the application of one or more external stimuli (e.g., light of a specific wavelength). In various embodiments, the terminal reactive moiety, the targeting moiety, and the affinity tag may be activatable (e.g., photoactivatable).

As used herein, the term “affinity tag” refers to a chemical moiety that may be attached to the disclosed bifunctional crosslinker to facilitate enrichment of crosslinker modified species from a sample. Similarly, a target protein may naturally express or be engineered to express an affinity tag and the crosslinked protein may be enriched using the affinity tag.

As used herein the term “ambient conditions” refers to conditions generally present in a clinical or laboratory setting. Thus, ambient conditions include a pH of about 6 to about 8 and temperature ranging from about 0° C. to about 37° C.

As used herein, the term “appended moiety” refers to a chemical species that is attached to the basic bifunctional crosslinker structure (i.e., the internal linker portion plus the matching terminal reactive groups) to enhance performance of the bifunctional crosslinker. Thus, an appended moiety may include, without exception, a targeting moiety, a cell permeability-enhancing moiety, and an affinity tag. Appended moieties may be directly attached to the bifunctional crosslinker. Alternatively, appended moieties may be attached to the bifunctional crosslinker through a linker, which may or may not include one or more selective cleavage sites or cleavage sequences according to the particular requirements for the bifunctional crosslinker.

As used herein the terms “capping” and “capped” of a functional or reactive group refers to a grouping of atoms that when attached to a functional or reactive group in a molecule masks, reduces, or prevents that functionality or reactivity. Thus, a in the context of the present disclosure, a reactive group such as a thiol may be capped by reaction with haloacylamides, maleiimides or another thiol reactive agents to prevent further reaction or oxidation of the capped reactive group.

As used herein the term “cell permeability” refers to the ability of an agent to traverse the cell membrane of an intact cell under physiological conditions without the aid of cell permealizing agents. Cell permeation in live eukaryotic cells occurs through endocytosis, which may comprise passive transport or active transport (e.g., receptor mediated endocytosis or pinocytosis).

As used herein the term “cell permeability-enhancing moiety” refers to any moiety that may be appended to the disclosed bifunctional crosslinker that enhances cell permeability. Cell permeability-enhancing moieties may include, for example, a peptide sequence containing predominantly hydrophobic or hydrophilic amino acids.

As used herein, the term “cleavage agent” generally refers to agents that split a complex molecule (e.g., a protein, a bifunctional crosslinker, or a linker that tethers an appended moiety to the bifunctional crosslinker) into multiple simpler molecules, whether through enzyme-mediated, chemical-mediated or photochemical mediated hydrolysis or reduction of covalent bonds (e.g., disulfide bonds, diols, or ester bonds), oxidation or other means.

As used herein the phrase “effector agent” refers to any agent that may be contacted with a sample comprising a cell population for determining the affect that the agent has on the protein-protein interactions of the sample. Thus, effector agents may include putative or known activators or inhibitors of specific proteins or protein complexes.

As used herein the term “enrichment” refers to techniques that increase the relative proportion of a species (e.g., particular polypeptide sequence bound to a bifunctional crosslinker or terminal portion of a bifunctional crosslinker) within a sample. Illustrative techniques for enriching a particular species in a sample may include, without limitation, electrophoresis (e.g., SDS PAGE), or chromatography (e.g., affinity chromatography or HPLC).

As used herein, the term “hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K), and Arg (R).

As used herein, the phrase “hydrophobic amino acid” refers to an amino acid exhibiting a hydrophobicity of greater than zero according to the normalized consensus hydrophobicity scale of Eisenberg, 1984, J. Mol. Biol. 179:125-142. Genetically encoded hydrophobic amino acids include Pro (P), Ile (I), Phe (F), Val (V), Leu (L), Trp (W), Met (M), Ala (A), Gly (G), and Tyr (Y).

As used herein, the term “in situ” generally refers to an event occurring within a prokaryotic cell or a eukaryotic cell. Thus, in situ analysis protein interactions describes analysis of proteins or protein complexes located within a whole cell, whether the cell membrane is fully intact or partially intact where protein contents remain within the cell. In situ analysis of protein interactions may be performed on cells derived from a variety of sources, including an organism, an organ, tissue sample, or a cell culture. Moreover, the methods disclosed herein may be employed to analyze protein interactions in situ in cell samples that are fixed or unfixed where the proteins or protein complexes are located within a cell.

As used herein the terms “isotopic labeling variant” and “isotopic variant” generally refer to a member of a family of chemical entities that are substantially chemically identical, but which are distinguishable by mass because of the presence of a different isotope. In some embodiments, preferred isotopic labeling variants do not change the elution profile of the variant species.

As used herein the phrase “linker cleavage site” refers to the portion of the bifunctional crosslinker capable of chemical cleavage or enzymatic cleavage under predetermined conditions.

As used herein, the term “mass spectrometry” and the abbreviation “MS,” generally refer to analytical techniques in which individual molecules are converted into ions (i.e., electrically charged molecules) followed by detection of the mass/charge ratios of the ionized species. Illustrative mass spectrometry techniques include, without limitation, MALDI-TOF-MS, ESI-MS, LC-ESI-MS, MALDI-TOF/TOF, ESI-MS-MS, FAB-MS, or FTICR-MS. In the practice of the disclosed methods, specific MS analytical techniques may be used singly or in combination with other MS techniques.

As used herein, the phrase “mass unit differential” refers to a detectable mass difference for two members of an isotopic labeling pair, which may be determined using MS techniques. In some embodiments, a mass unit differential as small as 1 Da may be detected for a bifunctional crosslinker.

As used herein, the term “mass-shifting variant” refers to the member of a differentially labeled bifunctional crosslinker set that is substantially identical with its mate or mates that is labeled with the heavier or heaviest isotopic label.

As used herein the term “physiological conditions” refers to conditions generally present in a mammalian body. Thus, physiological conditions mean a pH of about 6.5 to about 7.5 and temperature ranging from about 25° C. to about 37° C.

A “protein fragment” refers to an amino acid sequence derived from a target protein or protein complex that contains at least about 3-10 amino acids. However, in some embodiments, the protein fragment contains about 10-20 amino acids, and in still other embodiments, the protein fragment contains at least about 20-50 amino acids derived from the specified protein or protein complex.

As used herein, the term “proteolytic cleavage sequence” refers to amino acid sequences that are preferentially cleaved by a specific proteolytic enzyme. Useful proteolytic cleavage sequences include amino acid sequences that are recognized and cleaved by proteolytic enzymes such as trypsin, plasmin, or enterokinase K.

As used herein, the term “specific cleavage” refers to the ability of a cleavage agent to cleave a particular cleavage sequence and not other sequences. Thus, the disclosed bifunctional crosslinker may be designed to include specific sequences in a linker portion that are not present in a target protein or protein complex.

As used herein, the term “selective binding” means the ability of a moiety to bind its target in a specific manner. A target molecule should have an intrinsic equilibrium association constant (KA) for its target moiety no lower than about 10⁵ M−¹ under physiological conditions.

As used herein, the term “symmetrical” regarding the symmetrical bifunctional crosslinker, means that the linker includes an internal axis at or near the center of the crosslinker with matching terminal moieties at each end, and matching internal linker portions positioned on either side of the internal axis.

As used herein, the term “targeting moiety” refers to a chemical species (i.e., an organic molecule or a biomolecule) that recognizes and specifically binds to one or more proteins in a protein-protein complex. In some embodiments, the target moiety is capable of binding to protein fragments near or adjacent to the portions that transiently interact (e.g., interact only for seconds or minutes).

As used herein, the term “target protein” refers to the protein or protein fragments (i.e., the portions of a protein that remains bound to a bifunctional crosslinker following a cleavage step) that a bifunctional crosslinker binds, whether the target protein has been identified as a protein of interest before the analysis or is an anonymous protein (i.e., not known or not known to associate with another protein or protein complex that is ultimately detected using MS analysis). Although a target protein or protein fragment may be anonymous, in those embodiments where the bifunctional crosslinker includes a targeting moiety, the target protein or protein fragment may be identified or may be an unidentified protein or protein fragment that demonstrates homology to the protein or protein fragment to which the targeting moiety is designed to bind specifically. A target protein or protein fragment may contain an affinity tag, which may be naturally expressed or engineered, that may facilitate enrichment of the protein or protein fragment.

As used herein the term “terminal reactive group” generally refers to a reactive moiety on a crosslinker that can react with a functionality on the target protein or protein-protein complex resulting in target protein or protein complex binding to the bifunctional crosslinker.

II. Agents for Analysis of Protein Interactions.

Provided herein are substantially symmetrical, cleavable, isotopically labeled bifunctional crosslinkers that are useful for analyzing protein interactions as well as relative protein-protein interactions between samples. In some embodiments, the bifunctional crosslinker is a substantially linear molecule with paired reactive terminal moieties positioned symmetrically at the terminal ends of the bifunctional crosslinker. A representative bifunctional crosslinker is shown below, where n is 1-6 and m is 2-12.

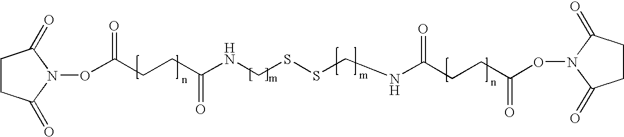

The terminal reactive group may bind to the target protein or protein complex covalently or non-covalently, in either instance, the binding should have an affinity sufficient for the resultant bifunctional crosslinker-protein complex to remain bound during the subsequent processing steps. Thus, in some embodiments where the reactive group is non-covalently bound, the Ka of the terminal reactive group to the target protein or protein complex occurs with an affinity greater than 10⁹ M−¹. In some other embodiments, the Ka of the terminal reactive group to the target protein or protein complex occurs with an affinity greater than 10¹² M⁻¹. The terminal reactive group may react with functional groups found in proteins, for example NH₂, SH, or COOH groups to form covalent bonds. In some embodiments, the terminal reactive group may be spontaneously reactive. Examples of spontaneously reactive moieties include but are not limited to, reactive esters, maleiamide or derivatives, iodoacetamide. In some embodiments, the terminal reactive group is activatable, e.g. an aryl azide which is activated with UV light.

Representative terminal reactive moieties may include aldehydes, acids, imidoesters, N-hydroxysuccinimide esters, aryl azides, difluorobenzenes, aryl halides, carbodimides, haloacetyls, iodoacetyl groups, pyridyl disulfides, hydrazides, isocyanates, or maleimides. In some specific embodiments, the terminal reactive moieties comprise amine reactive groups capable of binding to lysine residues in a polypeptide.

The bifunctional crosslinkers include an internal linker portion disposed between the matching terminal reactive moieties. The internal linker portion is substantially symmetrical about its central internal axis. The internal linker portion includes at least one cleavage site and is generally devoid of any charges unless specifically incorporated to aid in mass spectrometry or for cell permealization. In most circumstances, these charges will be incorporated in the appended moieties.

In some embodiments, the crosslinker comprises one or more cleavage sites positioned away from the terminal ends (e.g., in the internal linker portion or an optional linker that tethers an appended moiety to the bifunctional crosslinker). In some embodiments, the cleavage sites may be positioned at or near the internal axis of crosslinker. After enrichment (e.g., by SDS-PAGE or chromatography) of the cross-linked protein complex and before or after protein digestion but prior to MS, such cleavage sites may be used to cleave or separate the cross-linked proteins or cross-linked fragments into their individual components (either peptides or proteins depending on whether the cleavage occurred before or after trypsin digest). Such a cleavage step simplifies MS analysis and facilitates the identification of crosslinker-modified peptides in the MS spectra.

A bifunctional crosslinker may be designed to lack any protein sequences that are capable of being cleaved by one or more specific cleavage enzymes or lack any other cleavable moieties such as a disulfide linkage or a phosphodiester linkage. These linkers may be useful when purification of the cross-linked complex is not performed and it is necessary to distinguish, using MS, between two cross-linked peptides and simply a modified peptide (from a protein that reacted with the crosslinker but did not undergo intermolecular cross-linking with another protein).

In some embodiments, the linker cleavage site may be a single cleavage site positioned in the central axis of the internal linker portion so that cleavage yields two fragments. In other embodiments, the linkage cleavage site comprises more than one cleavage site such that cleavage yields two substantially similar terminal portions (each of which may be attached to portions of the target protein) and one or more residual central portions. By way of example, when the cleavage site comprises two cleavage sites, cleavage will yield two terminal portions and one residual central portion; and when the cleavage site comprises four cleavage sites, cleavage will yield two terminal portions and three residual central portions. In other embodiments, the linker cleavage site is disposed within a linker that appends a functional moiety (e.g., a cell permeability enhancing moiety, targeting moiety, or an affinity tag) to the bifunctional crosslinker.

In embodiments where the bifunctional crosslinker includes more than one cleavage site, an even number of the cleavage sites are positioned symmetrically about the internal axis, with the remaining site positioned at or near the central axis. This symmetrical configuration results in equal mass for each portion of the bifunctional crosslinker including a terminal moiety following cleavage and ensures that the mass differential results from the differential isotopic label rather than the presence of appended unbalanced moieties. In alternative embodiments, the cleave sites are randomly disclosed along the internal linker portion where each of the two cleavage sites closest to the terminal moieties are positioned equidistant from the termini, thus resulting in equal mass exclusive of the mass differential resulting from the isotopic variation.

Similarly, in embodiments where the bifunctional crosslinker includes a targeting moiety, the targeting moiety may be position off-center from the internal axis and the targeting moiety is mirrored by a matching targeting moiety, although this is not required when the appended moiety may be cleaved before the analysis step. This symmetrical configuration results in equal mass for each portion of the bifunctional crosslinker including the terminal moiety following cleavage and ensures that the mass differential results from the differential isotopic label rather than the presence of appended unbalanced moieties.

As is known in the art, specific bonds may be selectively cleaved by particular agents, for example: disulfide bonds may be selectively cleaved by reducing agents; diol bonds may be selectively cleaved by oxidizing agents; diazo bonds may be selectively cleaved by dithionites; alkyl sulfones, which may be selectively cleaved by bases; ester bonds, which may be selectively cleaved by acids, bases, or esterases; and peptide bonds may be selectively cleaved by particular proteases.

Enzymatic cleavage agents may include, for example, proteases that hydrolyze peptide bonds between amino acid residues in a polypeptide, phosphodiesterases that hydrolyze phosphodiester bonds, or lipases that hydrolyze esters. Chemical cleavage agents may include hydrolyzing chemicals such as acids, bases, periodate, dithionite, hydroxylamine, dithiothreitol (DTT), Tris-carboxyethylphosphine, or beta-mercaptoethanol (BME). In some embodiments, the site-specific cleavage agent may cleave both the protein sample and the bifunctional crosslinker. In other embodiments, the bifunctional crosslinker is designed to be resistant to cleavage by a specific cleavage agent. Accordingly, a sample-specific cleavage agent refers to a cleavage agent that cleaves the protein sample but not the bifunctional crosslinker (i.e., protein-sample specific). In other embodiments, the bifunctional crosslinker is designed to be susceptible to cleavage by a specific cleavage agent. Thus, a bifunctional-crosslinker-specific cleavage agent refers to cleavage agents capable of cleaving the bifunctional crosslinker but not the protein sample.

The internal linker portion of the bifunctional crosslinkers includes one or more isotope that may be used to distinguish a bifunctional crosslinker from a mass-shifting variant of the same bifunctional crosslinker. Exemplary differential isotopes that may be used to create an isotopic labeling pair may include, without limitation: carbon (C¹² and C¹³), nitrogen (e.g., N¹⁴ and N¹⁵), sulfur (e.g., S³² and S³⁴), oxygen (e.g., O¹⁶, O¹⁷ and O¹⁸), bromine (Br⁷⁹ and Br⁸¹), or chloride (e.g., Cl³⁵ and Cl³⁷) with the proviso that proteins or protein fragments with same sequences upon modification with these crosslinker pairs will coelute on sensitive chromatographic techniques such as HPLC. Therefore in all embodiments, deuterium (H²) and hydrogen are disfavored as isotopic labeling pairs because they do not co-elute when separated using sensitive chromatographic techniques (e.g., HPLC) and the pair members are susceptible to exchanging positions during processing and convolute MS analysis. It is further possible to mix and match different isotopes of different atoms to create crosslinker sets with more than 2 members, but same structure and elution profile. For example, one member of the crosslinker set containing at least two carbons and two oxygen atoms, may contain all atoms at natural abundance (i.e., predominantly two C¹²s and two O¹⁶s), another may contain two C¹³s and two O¹⁶S , a third one two C¹³ and one O¹⁶ and one O¹⁸ and a fourth one with two C¹³s and two O¹⁸s, giving a set of four crosslinkers each separated by 2 mass units from its next lower mass variant.

The bifunctional crosslinkers may vary in length. The length of a specific bifunctional crosslinker may be optimized for various performance characteristics depending upon the particular embodiment. Thus, linker length may be increased to increase the number of atoms available as isotope differentiating atoms between the pairs. For example, in embodiments where the isotope pair comprises C¹² and C¹³, the internal linker portion (i.e., the portions of the bifunctional crosslinker excluding the terminal reactive moieties) of the linker may comprise at least 2 carbon atoms, providing at least a 2 Da mass shift differential for a non-cleaved bifunctional crosslinker pair. In alternative embodiments, internal portion of the bifunctional crosslinker may comprise at least 4 carbon atoms, providing at least a 4 Da mass shift differential for the non-cleaved bifunctional crosslinker pair. Alternatively, the desired mass differential may arise from a combination of atoms. For example, a linker with one O¹⁸ and two C¹³ will provide the same mass differential as four C¹³s or two O¹⁸s.

Furthermore, the bifunctional crosslinker length may be varied to span a particular distance from terminal end to terminal end, to result in cross-linking protein fragments that are in close proximity (e.g., less than 4 angstroms) or farther proximity (e.g., more than 50 angstroms) to each other. The length of the bifunctional crosslinker may be optimized for a particular protein by screening a library of bifunctional crosslinker of various lengths.

In some embodiments, the mass unit differential for an intact bifunctional crosslinker pair is 12 Da and, consequently, a 6 Da mass unit differential for the portion of the symmetrical cleaved bifunctional crosslinker that remains attached to a target protein following cleavage. In other embodiments, the mass unit differential for a bifunctional crosslinker pair is an 8 Da mass unit differential for an intact bifunctional crosslinker and, consequently, a 4 Da mass unit differential for the symmetrical cleaved bifunctional crosslinker that remains attached to a target protein following cleavage. In other embodiments, the mass unit differential is 2 Da mass units for an intact bifunctional crosslinker and, consequently, a 1 Da mass unit differential for the symmetrical bifunctional crosslinker linker that remains attached to a target protein following cleavage.

Thus, an isotopic labeling pair may comprise a non-isotopic-labeled bifunctional crosslinker and an analogous isotope-labeled bifunctional crosslinker (i.e., a mass-shifting variant). Exemplary differential isotopes that may be used to create an isotopic labeling pair may include, without limitation: carbon: C¹² and C¹³; nitrogen: N¹⁴ and N¹⁵; sulfur: S³² and S³⁴; oxygen: O¹⁶, O¹⁷ and O¹⁸; Br⁷⁹ and Br⁸¹; or chloride: Cl³⁵ and Cl³⁷. Carbon, nitrogen, oxygen, and sulfur are preferred members of a labeling pair as they are not easily displaced. Other atoms with stable isotopes or other stable isotopes of the atoms described above may also be used as long as they satisfy the criteria of coelution of the identical modified sequences labeled or unlabeled with isotopes of the same set of atoms. Although it is theoretically possible to use radioisotope, they are not desirable due to radiation toxicity and contamination. Deuterium (H²) and hydrogen are disfavored as isotopic labeling pairs because they do not co-elute when separated using sensitive chromatographic techniques (e.g., HPLC) and the pair members are susceptible to exchanging positions during processing and convolute MS analysis.

In some embodiments, the crosslinker is capable of permeating the cell membrane of a substantially intact cell under predetermined conditions (e.g., physiological conditions). Bifunctional crosslinkers with enhanced cell permeability are particularly useful for methods of analyzing protein interactions for proteins or protein complexes present in a whole cell. In some embodiments, keeping the molecular weight of the crosslinker below 5000 Da may enhance permeability through passive diffusion mechanism. Permeability through passive diffusion mechanisms may also be enhanced by minimizing the number of charged groups present in the bifunctional crosslinker including the charge groups present in any appended moieties.

Cell permeability-enhancing moieties may be categorized according to the mechanism that is employed to introduce an agent into a cell. Agents may pass into an intact or substantially intact whole cell through passive transport (e.g., through partial solubilization of the cell membrane) or through active transport (e.g., receptor-mediated endocytosis). Thus, passive transport enhancing-moieties may include hydrophobic or hydrophilic moieties such as hydrocarbons or polyethylene glycol (e.g., by increasing hydrophobicity) while retaining solubility in water or water with less than 10% of an organic solvent (e.g., ethanol, DMSO). Furthermore, cell permeation through the passive transport mechanism may be enhanced by designing the bifunctional crosslinker to have a low molecular weight (i.e., less than about 5000 Da), appending hydrophobic groups (e.g., several hydrophobic amino acids), and minimizing the number of charged moieties on the bifunctional crosslinker.

For embodiments where the permeability through active transfer mechanisms is desired, a permeability-enhancing moiety may be appended to the bifunctional crosslinker (e.g., poly-Arg or peptide tags). In some specific embodiments, the permeability- enhancing moiety may comprise an internalization sequence. The internalization sequence may comprise the TAT peptide sequence or the Antp internalization sequence.

In another aspect, the invention provides a bifunctional crosslinker including a targeting moiety. Such targeting moieties may include one or more chemical species that specifically bind to a particular protein sequence, a portion of a protein or portions of two proteins placed in close proximity, for example a particular carbohydrate sequence. Targeting moieties may be used to increase detection sensitivity of various analytical techniques. A targeting moiety may optionally be attached to a bifunctional crosslinker to target a protein or a protein complex by specifically binding to a polypeptide sequence present in the target protein or protein complex. As with other appended moieties, the targeting moiety may be attached to the bifunctional crosslinker directly or through a linker that may be optionally cleaved after the targeting function is accomplished.

In some embodiments, a targeting moiety may be small molecular weight chemical structures that are known to, or may be found to, specifically bind the targeted protein or protein complex and are attached to the bifunctional crosslinkers through a linker that does not significantly affect its specific binding. Alternatively, the targeting moiety may also comprise a biomolecule such as DNA or RNA aptamers, peptides, antibodies, or antibody fragments that are specific for a target sequence. Targeting moieties may be directly attached to the bifunctional crosslinker or may be attached through a linker. Illustrative small molecular weight targeting moieties may include, without limitation, biotin, or a nickel complex.

In another aspect, the invention provides a bifunctional crosslinker including one or more affinity tags. As with other appended moieties, the affinity tag may be attached to the bifunctional crosslinker directly or through a linker. Illustrative examples of affinity tags may include, but are not limited to, amino acid sequences (e.g., polyhistidine or antibody fragments), small-molecules (e.g. biotin), nucleic acid sequences (e.g., DNA, RNA, or PNA), or a fluorescent tag capable of enriching a target chemical entity through affinity capture and improved detection.

In all embodiments, the disclosed bifunctional crosslinkers may be optimized for peptide ionization for particular MS techniques. Significant considerations for optimization include stability (e.g., resistance to fragmentation by the MS device) and detectability (e.g., not suppressed by competing normal peptides). Thus, in embodiments where the MS technique employs the positive ion mode, the number of negative charges is minimized. In some embodiments the bifunctional crosslinker is designed to resist no fragmentation resulting from the MS technique. Furthermore, when LCMS is employed the bifunctional crosslinker should be capable of elution from an LC column. In MALDI, the bifunctional crosslinker and unmodified tryptic peptides should interact with the matrix similarly in terms of solubility and dispersion in the matrix.

In another aspect the present invention provides kits comprising a bifunctional crosslinker pair packaged in one or more containers (e.g., vials) in solution or lyophilized (which may optionally include a separate container with an appropriate solution to solubilize the lyophilized agent). In some embodiments, the each member of the bifunctional crosslinker pair is packaged in separate containers. In other embodiments, kits may include process protocols as well as analysis software.

In yet another aspect, the present invention provides libraries of crosslinkers that can be prepared by varying one feature of the bifunctional crosslinker. For example, a library may comprise pool of bifunctional crosslinker comprising a single species of terminal active moieties and the linker length is varied. Alternatively, the bifunctional crosslinker in a pool may be varied by the inclusion or omission of one or more appended groups, such as an affinity tag, a targeting moiety, or a cell permeability-enhancing moiety. Other libraries may include crosslinkers of similar structure and lengths but different reactive groups. Thus, provided herein are multiple pools of bifunctional crosslinkers from which a particular bifunctional crosslinker demonstrating a desired functionality may be selected using standard screening techniques.

II. Methods for Analyzing Protein Interactions.

Using the disclosed methods, a protein or a protein complex may be analyzed to elucidate the interaction between proteins or protein complexes that may be used in combination with mass spectrometry.

Using the disclosed methods a variety of samples may be analyzed for protein-protein interactions. Specifically, the disclosed methods may be employed in vitro or in situ. In one series of embodiments, one or more protein sample is analyzed in vitro. The samples are obtained from cell lysate of cell culture or a clinically relevant tissue or serum sample compared to a control sample. In another series of embodiments, one or more protein sample is cross-linked inside substantially intact cells before isolation and subsequent analysis. Crosslinkers useful in the disclosed method may be defined by the structure below:
D1-------R5----------D2

Where D1 and D2 are reactive functional moieties independently selected from reactive esters, aryl azides and other photoreactive groups (e.g. psoralans, coumarins), haloacyl, carboxyl, disulfides, maleimides, hydrazides, aldehydes, glyoxals, imidoesters, or other known reactive moieties known in the art for use with proteins. R5 is a cleavable or non-cleavable organic moiety with 2 or more carbon atoms, optionally containing 1 or more heteroatoms selected from the group consisting of O, N, S, P, halogen, B, As or Se.

The methods disclosed herein are useful to analyze protein interactions in situ where the protein contents are located within cell, whether the cell membrane is fully intact or partially intact, so long as the protein contents remain within the cell whether the cell is derived from a variety of sources including an organism, an organ, tissue sample or a cell culture. Moreover, the methods disclosed herein may be employed to analyze protein-protein interaction in situ in samples that are fixed or unfixed so long as the majority of the target protein complex or protein remain located within the cell.

In one embodiment, the disclosed methods provide methods for detecting protein-protein interactions in a sample or several samples. In general, one of the two populations to be compared is reacted with a normal abundance cross-linking agent, and the other is reacted with an isotopically enriched cross-linking agent. The cross-linked populations are then mixed so that any further processing steps will effect the isotopically enriched population to the same extent as the normal abundance population. After processing the samples in order to generate a sample appropriate for MS analysis for detecting a protein interaction of interest (for example gel electrophoresis, and subsequent enzymatic digestion/reduction of a cross-linked product), a peptide that bears all or part of the crosslinker is identified in the mass spectrum. The isotopic enrichment is not necessary to identify the species of interest, but a fragment that bears at least part of the crosslinker must be identified. Thus, the differential analysis may be applied to a sample comprising a previously identified protein interaction or previously unidentified protein interactions. A crosslinker pair useful to practice the methods of disclosed invention is shown below:
D1--------R5---------D2
D1--------R6---------D2

Where D1 and D2 are reactive functional moieties independently selected from reactive esters, aryl azides, and other photoreactive groups (e.g. psoralans, coumarins), haloacyl, carboxyl, disulfides, maleimides, hydrazides, aldehydes, glyoxals, imidoesters, or other known reactive moieties known in the art for use with proteins. R5 is a an organic moiety with 2 or more carbon atoms, optionally containing 1 or more heteroatoms.

R6 is the same structure as R5 except some of the atoms in the structure are heavy isotopes of those atoms to provide a mass differential of at least 2 Da between the cross-linked protein complexes, proteins or protein fragments (after protein digestion or crosslinker cleavage) generated with the two crosslinkers if the substitution of heavy isotope for the light isotope does not affect the mobility of cross linked products on HPLC.

The disclosed methods may further include an enrichment step. The enrichment may occur at various steps in the analytical methods. Thus, in one embodiment, the enrichment step occurs following cross-linking and before mixing cross-linked protein or protein fragments. In a preferred embodiment, the enrichment step occurs following cross-linking and combining of a first and second sample. In still other embodiments, the enrichment step occurs following the cleavage step and before the MS analysis. Enrichment techniques may include reverse phase chromatography, high performance liquid chromatography, ion exchange chromatography, gel electrophoresis, affinity chromatography, and the like.

In embodiments where the bifunctional crosslinker comprises an affinity tag, the enrichment step may comprise adhering the crosslinker bound to a protein or a protein fragment to a substrate, for example chromatographic material or magnetic beads, followed by one or more optional washing steps and release of the crosslinker bound a protein or a protein fragment.

The methods disclosed herein may include one or more optional cleavage step or steps. The cleavage steps facilitate one or more of the processing steps and reduce the complexity of the sample before MS analysis. In some embodiments, the cleavage step entails cleavage of the internal linker portion following cross linking step to produce a partial bifunctional crosslinker attached to a portion of a target protein. In preferred embodiments, the bifunctional crosslinker components of the linker-protein cleavage product consist of a whole bifunctional crosslinker or a portion of the bifunctional crosslinker.

In other embodiments, a cleavage step comprises specific cleavage to remove an appended moiety such as a permeability enhancing moiety, a targeting moiety, or an affinity moiety to thereby remove an appended moiety after that specific moiety has been utilized in the workflow. In those embodiments where the bifunctional crosslinker comprises an affinity tag, the cleavage step may occur before or after the one or more enrichment steps.

The resultant processed sample or samples may be analyzed using a mass spectrometry technique including MALDI-TOF-MS, ESI-MS, LC-ESI-MS, MALDI-TOF/TOF, ESI-MS-MS, FAB-MS, or FTICR-MS. Specific MS analytical techniques may be used singly or in combination with other MS techniques.

Another aspect of the invention relates to qualitative and quantitative differential analysis of a protein or a protein complex using non-coded crosslinkers along with isotope-coded crosslinkers. A general scheme for protein analysis using the disclosed crosslinkers is set forth in FIG. 2. Thus, the disclosed methods are useful for identifying protein-protein interactions.

Additionally, the disclosed methods may be used to determine the relative amounts of a specific protein or proteins present in one or more complex samples. The measurement of the relative amounts of a protein-protein interaction in two sample populations (for example a sample from a diseased state as compared to a healthy state) depends on generating a basis for detection and differentiation. This is accomplished by cross-linking the protein-protein interaction in the two differing populations (e.g., a health sample and a disease sample or a sample contacted with an effector agent and a sample not contacted with the same effector agent) with cross-linking agents that chemically react in identical ways, but have mass differentiators in the form of differing stable isotope compositions.

The chosen chemical species bearing the crosslinker portion will have a series of masses characteristic of the isotopic composition associated with it. The isotopic composition includes contributions from the abundances of all the elements comprising that species. These include the normal abundance contributions from the peptide(s) involved and the crosslinker, and contributions from the isotopically labeled crosslinker. The mass spectrometer may resolve adjacent mass/charge values to make accurate determinations of relative amounts of the protein or protein fragments.

The measurements are based on the ability to determine the relative contributions of the isotope labeled and unlabeled crosslinker to the observed mass peaks that comprise the isotopic distribution of the peptide species derived from the cross-linked protein.

This determination is enhanced by employing a with as many atoms as is practical having a high degree of enrichment (e.g., a high percentage of 13C at each labeled carbon atom). An example is provided below that uses a cleavable crosslinker with an isotope enriched composition that results in a 4 Da mass shift per cleaved cross- linked peptide. The example will present several methods of determining the relative contributions of a peptide from the calmodulin-melittin cross-linked protein complex using an isotope labeled and unlabeled crosslinker pair.

### EXAMPLES

Practice of the invention will be still more fully understood from the following examples, which are presented herein for illustration only and should not be construed as limiting the invention in any way.

In the following experiments, 13C labeled succinic acid (¹³C₄) and adipic acid (¹³C₆) were obtained from Cambridge Isotopes and Aldrich. All other reagents were purchased from Aldrich. N-hydroxysuccinimidyl ester of trifluoroacetate (TFA-NHS) may be prepared by the method of Sakaibara & Inukai (Bull Chem Soc Jpn 1965, 38, 1979).

### Example 1

### Adipic Acid bis NHS ester

Adipic acid (1.0 g) was coevaporated with anhydrous dimethylformamide (DMF, 2×5 ml) and was redissolved in anhydrous DMF (10 ml). To this solution, diisopropylethylamine (3.93 ml, 3.3 equivalent) was added followed by the addition of TFA-NHS (4.33 g, 3.0 equivalent). The mixture was stirred at room temperature for 7 hours and then concentrated to dryness under high vacuum while keeping the temperature below 30° C. The residue was coevaporated with acetonitrile (1-2 times). Residue was mixed with dichloromethane (25 ml) and water (25 ml). After vigorous shaking, a white solid precipitated. The solid was filtered, washed with dichloromethane (2×4 ml), water (2×5 ml) and once again with dichloromethane. After drying, 1.94 g (83.4% yield) of white solid was obtained. Purity by reverse phase HPLC ˜90%; Proton NMR-DMSO-d⁶: 1.71 ppm, br. m, 4H, CH₂; 2.72 ppm, br. t, 4H, CH₂C(O); 2.8 ppm, br. s, 8H, NHS ester protons.

### Example 2

### ¹³C Labeled Adipic Acid (¹³C₆) bis NHS Ester

Adipic acid-¹³C₆ (100 mg) was coevaporated with anhydrous DMF (2×1 ml) and was redissolved in anhydrous DMF (1 ml). To this solution, diisopropylethylamine (4.4 equivalent) was added followed by the addition of TFA-NHS (4.0 equivalent). The mixture was stirred at room temperature for 7 hours and then concentrated to dryness under high vacuum while keeping the temperature below 30° C. The residue was coevaporated with acetonitrile (1-2 times). The residue was mixed with dichloromethane (2.5 ml) and water (2.5 ml). After stirring for a few minutes, a white solid precipitated. The solid was filtered, washed with dichloromethane (2×0.5 ml), water (2×0.55 ml) and once again with dichloromethane. After drying, 100 mg (44% yield) of white solid was obtained. Purity by reverse phase HPLC ˜90%; Proton NMR-DMSO-d⁶: 1.72 ppm, d of br. s, 4H, ¹J_{13C,H} 125 Hz, CH₂; 2.75 ppm, doublet of br. m, ¹J_{13C,H} 124 Hz, 4H, CH₂C(O); 2.82 ppm, br. s, 8H, NHS protons. MS: [M+Na]⁺ 369.21.

### Example 3

### DisuccinylCystamine-bis NHS Ester (FIG. 3)

Step 1: Synthesis of S-tritylcysteamine: Cysteamine-HCl (4.00 g) was dissolved in trifluoroacetic acid (10 ml). To this triphenylcarbinol (8.89 g, 0.97 equivalent) was added and mixture was stirred at room temperature for 3 hours. The resultant dark red solution was poured over 400 ml of deionized water with vigorous stirring. A white solid precipitated. The aqueous suspension was made basic by addition of either DIEA or triethylamine (TEA). The white solid was filtered, washed with alkaline water (made alkaline with DIEA or TEA, 2×100 ml). The residue was coevaporated with acetonitrile (3×50 ml) to remove remaining water. Yield: quantitative. Proton NMR: 2.26 ppm, t, 2H, S CH₂; 2.51 ppm, t, 2H, N CH₂, 7.25 & 7.35 ppm, m, 15H, trityl.

Step 2: Synthesis of S-Trityl-N-succinylcysteamine. S-Tritylcysteamine (10.00 g) was coevaporated with anhydrous DMF (3×25 ml) and then redissolved in anhydrous DMF (35 ml). To this DIEA (27.8 ml, 5 equivalent) and succinic anhydride (3.50 g, 1.1 equivalent) were added and mixture was stirred at room temperature overnight. Reaction mixture was concentrated and coevaporated with acetonitrile (3×25 ml). The residue was mixed with DCM (125 ml) and water (125 ml) and stirred at room temperature for few minutes. Organic layer was separated and washed with water (2×125 ml). After drying over anhydrous Na₂SO₄, a white solid separated. It was filtered washed with acetonitrile and DCM. Organic filtrate was concentrated to dryness and resuspended in diethyl ether. Another crop of slightly off-white was obtained. Proton NMR of both solids was identical. Surprisingly, free acid rather than salt was obtained. Total yield: 10.71 g (81.5%); proton NMR: 2.18 ppm, t, 2H, SCH₂; 2.24 ppm and 2.37 ppm, triplets, 4H, CH₂C(O)NH and CH₂C(O)OH, 2.95 ppm, q, 2H, N CH₂, 7.23 ppm & 7.31 ppm, m, 15H, aromatic, 7.92 ppm, t, 1H, NH and 12.03 ppm, br. s, 1H, COOH.

Step 3: Synthesis of Disuccinlycystamine. S-Trityl-N-succinylcysteamine (10.7 g) was suspended in a mixture of water and acetic acid (4:1, 125 ml). To this 12 (6.44 g, 1 equivalent) was added and mixture was stirred vigorously at room temperature. Starting material slowly dissolves, but before all of it dissolves, product starts precipitating. After 6.5 hours, the solid was filtered, washed with water (2×25 ml), CCl₄ (5×25 ml) and acetonitrile (100 ml). Product was dried in air to give 3.24 g (72.1%) of white solid. Proton NMR: 2.31 ppm & 2.42 ppm, 2t, 8H, CH₂C(O)NH and CH₂C(O)OH, 2.75 ppm, t, 4H, CH2S, 3.30 ppm, q partially masked by water peak in DMSO-d6, 4H, 8.06 ppm, t, 2H, NH & 12.08 ppm, s, 2H, COOH. MS: [M+Na]hu + 375.11.

Step 4: Disuccinylcystamine-bis NHS ester formation. Disuccinylcystamine (200 mg) was coevaporated with anhydrous DMF (2×2 ml) and redissolved in anhydrous DMF (2 ml). To this solution, N-hydroxysuccinimide (156 mg, 2.4 equivalent) and N-ethyl-N-(3-dimethylaminopropyl)carbodimide (240 mg, 2.2 equivalent) were added and the mixture was stirred at room temperature overnight. The mixture was then concentrated to remove most of the DMF and was then coevaporated with acetonitrile (3×) to remove the remaining DMF. The residue was purified on a silica gel column using a step-wise gradient. Yield: 118 mg, (38%). Proton NMR: 2.58 ppm, t, 4H, CH₂, 2.78 ppm, s & 2.81 ppm, t, 12H, NHS ester protons & S CH₂, 2.90 ppm, t, 4H, CH₂, 3.47 ppm, q, 4H, N CH₂ & 7.98 ppm, br. t, 2H, NH, peaks at 5.47 ppm and 2.61 ppm due to some free NHS impurity (0.75 molar equivalent) were also present. MS: [M+H]⁺ 547.1 Da.

### Example 4

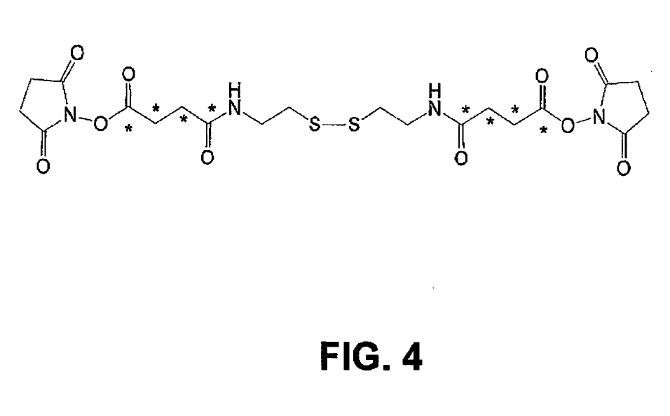

### ¹³C labeled Disuccinylcystamine (¹³C₈) bis NHS Ester (FIG. 4)

Step 1: Synthesis of ¹³C-labeled succinic anhydride (¹³C₄) and ¹³C-labeled Disuccinylcystamine (¹³C₈) bis NHS ester. Succinic acid-¹³C₄ was mixed with acetyl chloride (5 ml) and was heated at reflux in dry atmosphere until all solid went into solution. The mixture was cooled to room temperature and stored at 4 C overnight. Fine needles that crystallized were filtered and washed with diethyl ether. More solid came out in the filtrate, which was refiltered and washed with ether. The combined solid was dried in vacuo to yield 1.45 g (90.1%) of product. The remaining procedure was same as described in Example 3 steps 2-4 Disuccinylcystamine-¹³C₈-bis NHS ester: Proton NMR: 2.57 ppm, 4H, ¹J_{13C,H} 126 Hz, CH₂, 2.78 ppm, s, 8H, NHS ester, 2.80 ppm, t, 4H, SCH₂, 2.89 ppm, d of m, 4H, ¹J_{13C,H} 123 Hz, CH₂, 3.47 ppm, d of q, 4H, ²J_{13C,H} 4 Hz, N CH₂, 6.80 ppm, br. s, 2H, NH, peaks at 5.47 ppm and 2.61 ppm due to some free NHS impurity (0.62 molar equivalent) were also present. MS: [M+H]⁺ 555.1 Da.

### Example 5

### Cross-linking A Protein Complex

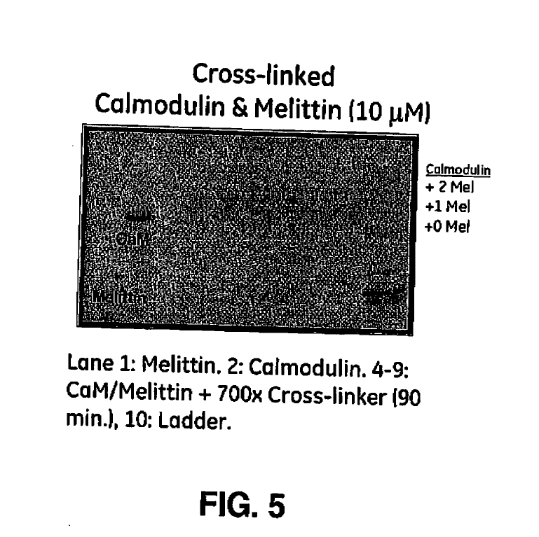

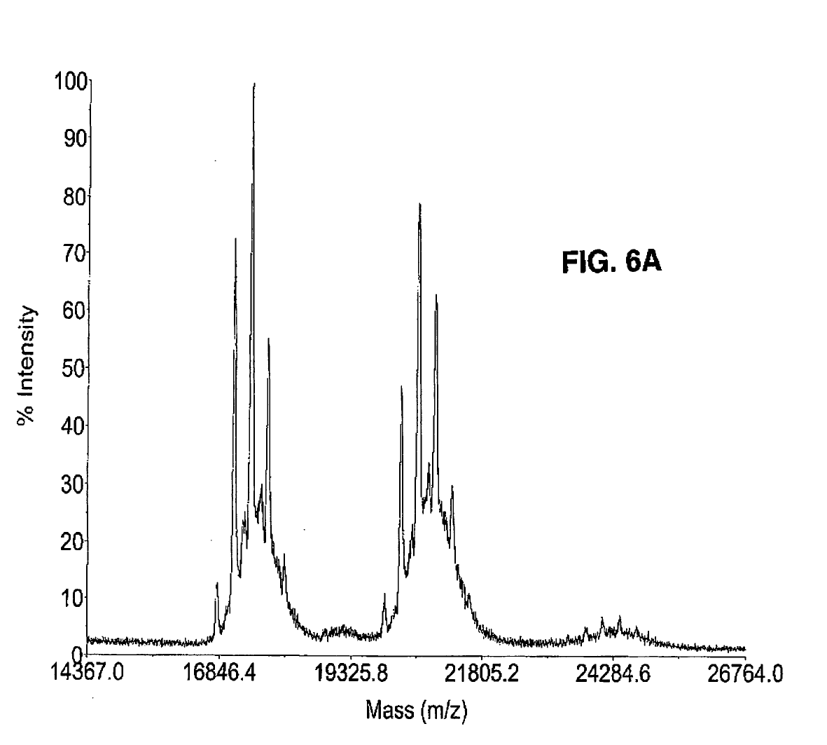

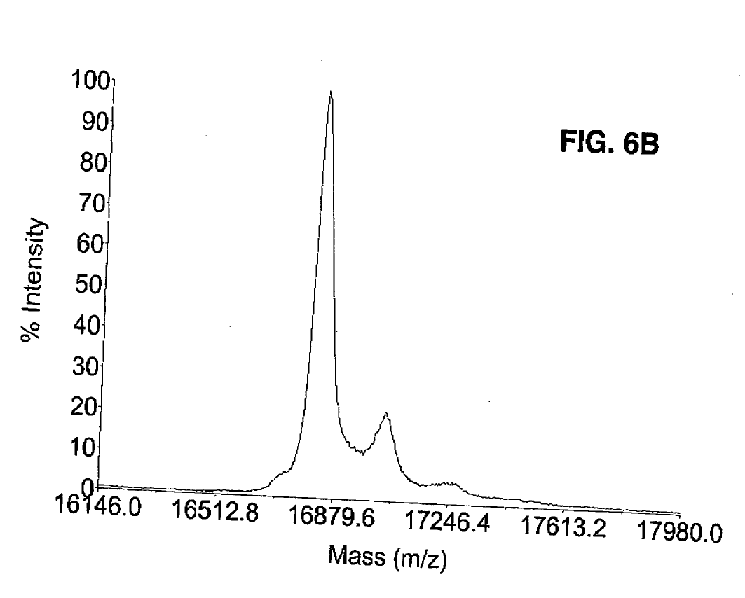

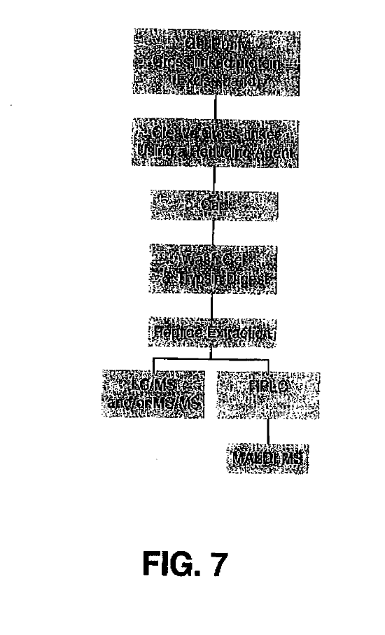

A model protein complex (bovine calmodulin, melittin, 10 uM each) was reacted with 100-700 molar equivalents of cross-linking reagent (Disuccinylcystamine-bis NHS ester) in 100-1000 μl of 20 mM Hepes buffer (pH 7.4), 1 mM CaCl₂ and incubated for 30-90 min at room temperature on a rocking platform. The progress of the cross-linking reaction was monitored by removing aliquots of the reaction mixture at times=0, 15, 30, 60, and 90 min., and quenching the NHS ester coupling reaction by adding an excess of a primary amine containing buffer (e.g., 20 mM Tris-HCl or NH₄HCO₃) and incubating at room temperature for 15 minutes. The reaction products were subsequently analyzed by SDS-PAGE (t=90 min, FIG. 5) and MALDI-MS (FIG. 6). Based on Coomassie staining, at least a 70% yield of cross-linked calmodulin-melittin was obtained following 90 minutes incubation. MALDI-MS analysis of the intact protein complex indicated both intramolecular and intermolecular bifunctional cross-linking (FIG. 6).

### Example 6

### Sample Preparation for MS Analysis

To simplify subsequent MS analyses, the cross-linked complex corresponding to a 1:1 ratio of calmodulin and melittin was gel purified from the reaction mixture. The protein band corresponding to the bifunctional crosslinker cross-linked CaM/Mel was excised from the gel, destained, and reduced with 25 mM dithiothreitol (in 25mM (NH₄)HCO₃) for 20 minutes at 56° C. to cleave the internal disulfide linkage in the bifunctional crosslinker reagent. Excess buffer was removed and 55 mM iodoacetamide in 25 mM (NH₄)HCO₃ was added to alkylate free sulfhydryl groups (20 minutes, 25° C.) and thus prevent the re-oxidation thiols.

Gel slices were washed, dried, and resuspended in 25 mM (NH₄)HCO₃/3% ACN [pH ˜8.5] containing 20 ng/μl of trypsin, and incubated at 37° C. for 16-24 hours. Digested peptides were gel-extracted and purified with a ZipTip-C18 column (Millipore) prior to MALDI-MS analysis. Extracted peptides were also analyzed by LC-MS, without Zip-tip clean up (See FIG. 17).

### Example 7

### In Silico Analysis

A predictive mass list was generated using external (MS-Digest) and internal (GE-ISD) in silco digest software tools to obtain the expected masses of trypsin digested calmodulin and melittin. As reported previously, CaM is N-terminal acetylated and contains a trimethylation modification to Lysine 115. (See, Schulz, D. M., et al., Biochemistry, 2004. 43(16): pp. 4703-15.) The melittin obtained (Source) was amidated at the C-terminus. These modifications, along with the possible oxidation of methionine residues and up to two missed cleavages resulting from putative incomplete trypsin proteolysis were used to generate the predictive mass list for these samples. For cross-linked samples, an additional mass shift of 319 Da was expected for cross-linked peptides, and 336 Da for peptides containing a partially hydrolyzed crosslinker.

To simplify the MS analysis, the bifunctional crosslinker is cleaved by reduction and re-oxidation is prevented by subsequent alkylation using iodoacetamide, corresponding to a mass shift of 216 Da per linker-peptide combination.

### Example 8

### Peptide identification by MALDI-MS

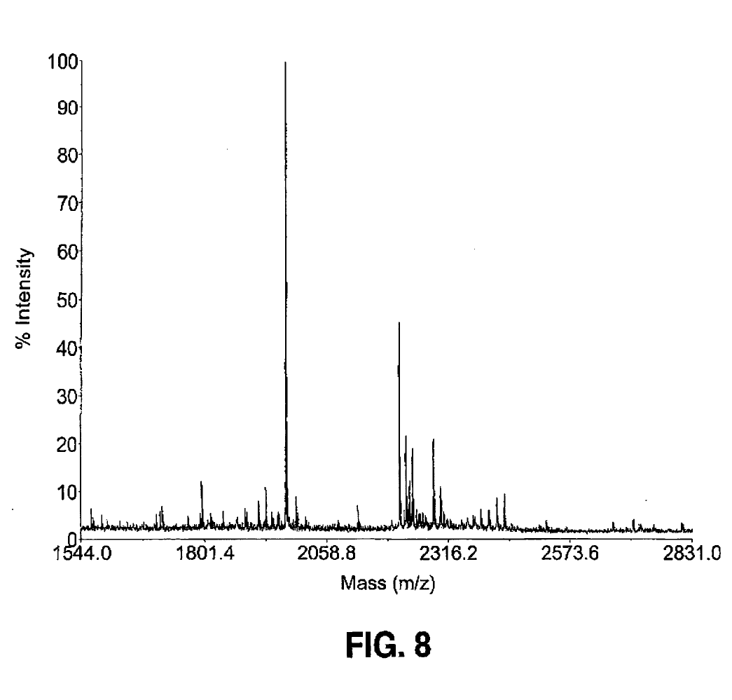

Peptide fragments for both calmodulin and melittin were individually identified by either MALDI-MS or ESI-MS. In addition, bifunctional crosslinker modified calmodulin peptides were identified by comparison of the predictive peptide mass list to the experimental MALDI-MS spectra. As shown in FIG. 8, the resultant MS data matched the predicated values as follows: expected m/z: 1970.93 (monoisotopic peak), observed m/z: 1970.92 (monoisotopic peak).

### Example 9

### Differential Analysis Using an Isotopically-label Pair of bifunctional Crosslinkers

To demonstrate the feasibility of differential expression analysis of protein complexes, the CaM/Mel complex (10 μM in 100 μl of 20 mM Hepes buffer, pH 7.4, 1 mM CaCl₂) was labeled separately with an equal quantity of the bifunctional crosslinker (Disuccinylcystamine-bis NHS ester) and the mass shifting variant (Disuccinylcystamine-¹³C₈-bis NHS ester) reagents (7 mM) and incubated at 25° C. for 90 minutes on a rocking platform. The reactions were subsequently quenched as described earlier in Example 5 and a 1:1 mixture (v/v) of the bifunctional crosslinker and bifunctional crosslinker mass-shifting variant cross-linked Cal/Mel was analyzed by SDS-PAGE. The cross-linked, isotopically labeled bifunctional crosslinker/peptide mixture was reduced, alkylated, and enzymatically digested following the same methods as described in Example 6. MALDI-MS analysis of the isotopically labeled bifunctional crosslinker/peptide mixture displayed a characteristic doublet with a mass shift=4 Da, due to the four ¹³C labels on the cleaved bifunctional crosslinker mass-shifting variant reagent.

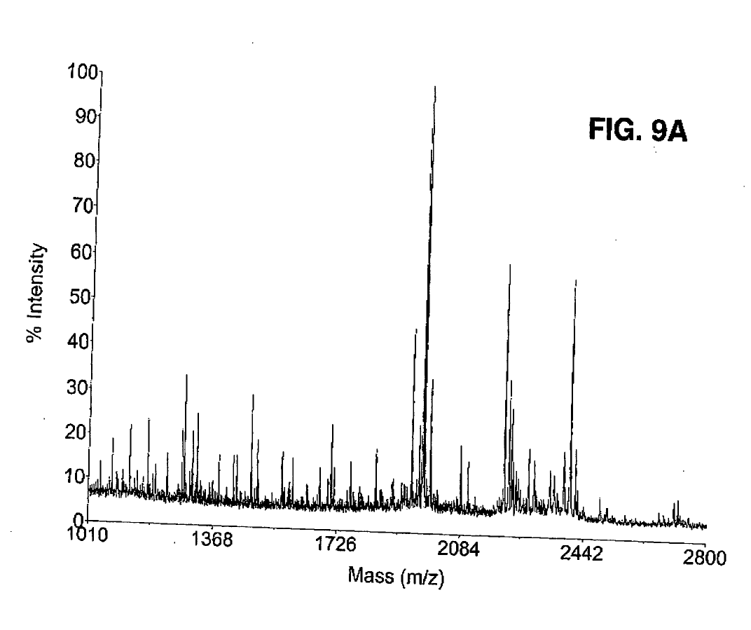

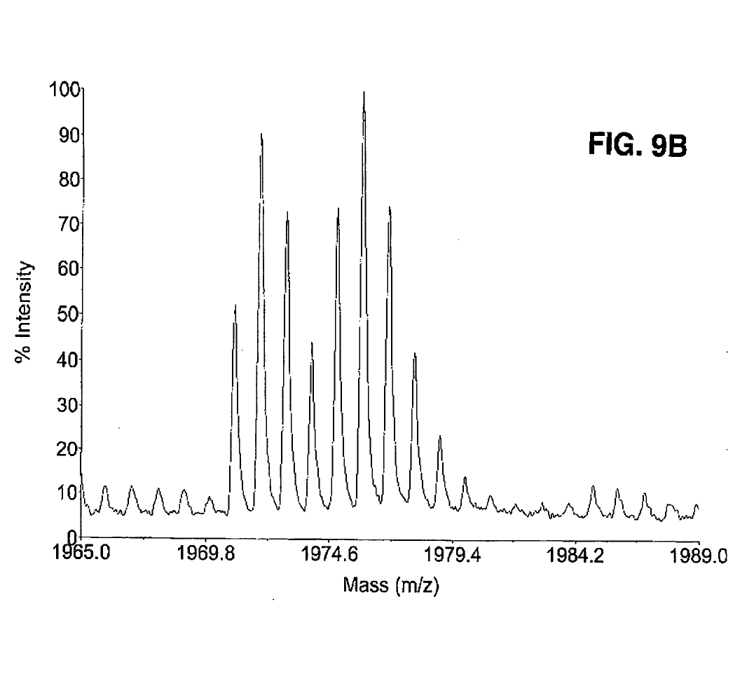

FIG. 9A depicts a MALDI-MS spectrum with signals at m/z 1970.9 and 1974.9, corresponding to the isotope labeled and unlabeled bifunctional crosslinker modified calmodulin peptide (residues 92-107). Ion intensities are consistent with the equal quantity of the CaM/Mel starting material used in both the isotope-labeled and unlabeled cross-linking reactions. FIG. 9B shows an expanded view of this region of the MS spectra, highlighting the isotopic pattern for both the isotope labeled and unlabeled crosslinker-modified peptide.

### Example 10

### Use of Calibration Curve Generated with Known Ratios

A linear regression analysis is performed with known ratios and measured peak areas for a specific peak from the isotope-labeled and unlabeled crosslinker modified peptides. An advantage of this method is that possible systematic matrix errors may be corrected. The disadvantage is that more samples need to be analyzed. A major requirement is that one must be able to calculate the fraction of overlap (if present) of the enriched isotopic peak chosen by the normal isotopic abundance. If there is no overlap, direct comparison is possible. For the 2 kDa peptide evaluated in this example, several peaks of the unlabeled peptide are independent of the isotope-labeled peptide, but the isotope-labeled peaks contain some contribution from the unlabeled peptide.

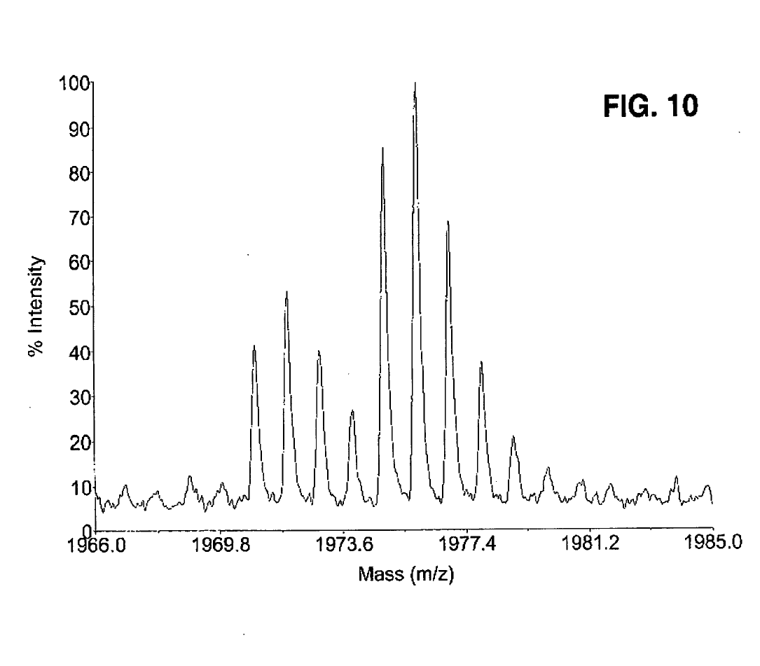

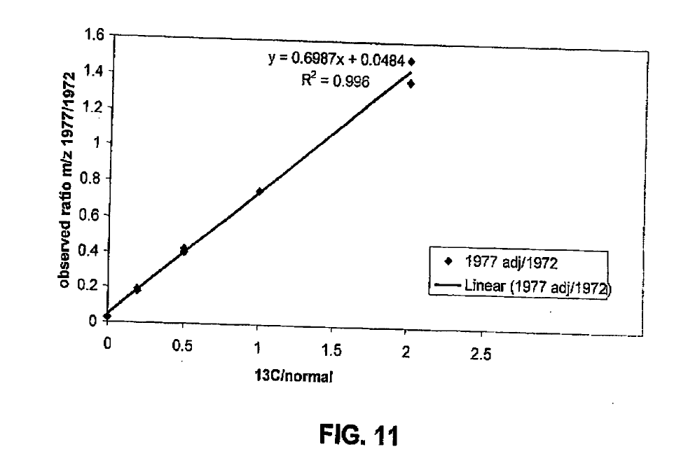

The pure normal abundance peptide is measured as part of the series of known ratios. The data from its pattern allows the calculation of its contribution to any of the peaks of the isotope distribution of the enriched mixture. In this example, the disuccinylcystamine-bis NHS ester (unlabeled) cross-linked calmodulin/melittin was mixed with twice the amount of the disuccinylcystamine-¹³C₈-bis NHS ester (isotope-labeled) cross-linked CaM/melittin. Following sample preparation as outlined in Example 6, MALDI-MS analysis (FIG. 10) of the modified Calmodulin peptide 92-107, showed the expected isotope pattern for both the isotope labeled and unlabeled peptide. The peak at 1972 Da was chosen as representative of the unlabeled species and 1977 Da was chosen as representative of the isotope labeled species. 1972 Da was chosen because of its larger relative area (compared with 1971 Da, for example) and 1977 Da was chosen as a compromise between relative peak area and degree of overlap with unlabeled species. The isotope labeled peak area at 1977 Da is adjusted by the known amount due to unlabeled species abundance. The ratio of the 1977-adjusted labeled peak area to the unlabeled peak is plotted versus the ratio of the amounts of isotopically labeled bifunctional/peptide mixture. A calibration curve generated from several ratios is given in FIG. 11. Any unknown ratio of 1977 (adjusted)/1972 could then be measured from this regression.

### Example 13

### Method of isotope dilution

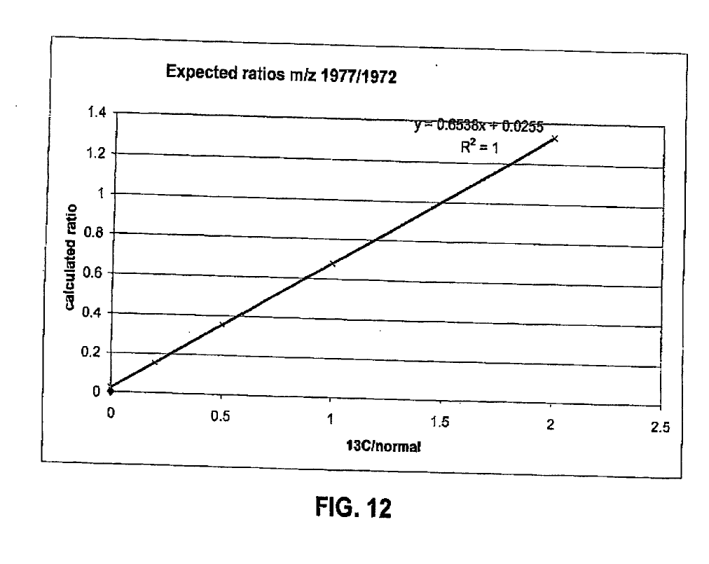

In this method, the expected calibration curve is calculated by performing a linear combination of the peaks from unlabeled peptide with the pure, isotope-labeled peptide. As such, any degree of overlap is acceptable, as long as the pattern difference can be differentiated. This method only requires the two standard measurements at the normal and enriched states, and assumes linearity. The contributions at each mass are calculated as mole fractional contributions. The mathematical treatment for this method can be found in various sources (e.g., 1995 IUPAC Pure and Applied Chemistry 67, 1943-1949). With the current data, the expected ratios can be plotted as shown in FIG. 12. The measurements performed as calibration points for method 1 can serve as test unknowns for method 2.

### Example 14

### Alternative Method to Isotope Dilution

A third method that can be used is a variant of the isotope dilution method. In this method only the isotope-labeled and the unlabeled need to be evaluated. From their isotopic patterns, it is possible to calculate the expected isotopic patterns of the respective crosslinked peptides of interest, and then the rest of the isotope dilution calculations can be performed. This method carries the significant risk that the peptide ionization will not be identical to the small molecule ionization. Some small molecules in MALDI mass spectrometry show some M+ besides the normal M+H+. Peptides almost exclusively show M+H+. This discrepancy would introduce some additional error.

The invention may be embodied in other specific forms without departing from the spirit or essential characteristics thereof. The foregoing embodiments are thereof to be considered in all respects illustrative rather than limiting on the invention described herein. The scope of the invention is thus indicated by the appended claims rather than by the foregoing description, and all changes that come within the meaning and range of equivalency of the claims are therefore intended to be embraced therein.

We claim:

1. A symmetrical bifunctional cross-linking agent, comprising a pair of reactive terminal moieties positioned at opposite ends of a cleavable, isotopically labeled internal linker portion.

2. The bifunctional crosslinker of claim 1, wherein each of the reactive terminal moieties comprises the same reactive group selected from N-hydroxysuccinimide ester, aldehyde, acid, imidoester, aryl azide, difluorobenzene, aryl halide, carbodimide, haloacetyls, pyridyl disulfides, hydrazides, isocyanate, or maleimide.

3. The bifunctional crosslinker of claim 1, wherein the internal linker portion comprises one or more cleavage sequences capable of cleavage by a chemical cleavage agent or an enzymatic cleavage agent.

4. The bifunctional crosslinker of claim 3, wherein the internal linker portion comprises an even number of paired matching cleavage sites, each paired cleavage site being positioned along the internal linker portion equidistant from the nearer terminal reactive moiety.

5. The bifunctional crosslinker of claim 3, wherein the internal linker portion comprises an odd number of cleavage sites, wherein one of the cleavage sites is positioned at or near the internal axis of the internal linker portion and the remaining cleavage site comprise paired matching cleavage sites, each paired cleavage site being positioned along the internal linker portion equidistant the nearer terminal reactive moiety.

6. The bifunctional crosslinker of claim 1, wherein the bifunctional crosslinker has a molecular weight between about 100 Da to about 5000 Da.

7. The bifunctional crosslinker of claim 6, wherein the molecular weight of the bifunctional crosslinker between about 100 Da to about 1000 Da.

8. The bifunctional crosslinker of claim 1, wherein the isotopic label comprises C¹³, N¹⁵, O¹⁷, O¹⁸; S³⁴, Cl³⁷and Br⁸¹.

9. The bifunctional crosslinker of claim 8, wherein the isotopic label is selected from C¹³, N¹⁵, O¹⁷, and O¹⁸.

10. The bifunctional crosslinker of claim 1, further comprising an affinity tag.

11. The bifunctional crosslinker of claim 10, wherein the affinity tag comprises a small molecule.

12. The bifunctional crosslinker of claim 10, wherein the affinity tag comprises amino acid residues or nucleic acid residues.

13. The bifunctional crosslinker of claim 12, wherein the nucleic acid residues comprise DNA, RNA, or PNA residues.

14. The bifunctional crosslinker of claim 1, further comprising a targeting moiety.

15. The bifunctional crosslinker of claim 14, wherein the targeting moiety comprises amino acid residues or nucleic acid residues.

16. The bifunctional crosslinker of claim 14, wherein the targeting moiety comprises a small molecule.

17. A bifunctional crosslinker, wherein the internal linker portion and the terminal reactive moieties comprise: 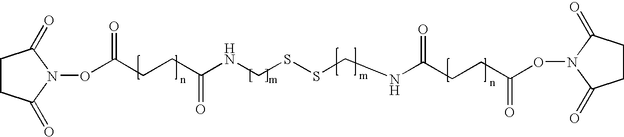
where n is 1-6, and m is 2-12.

18. The bifunctional crosslinker of claim 17, wherein one or more of the atoms comprising the internal linker portion is labeled with an isotope selected from C¹², C¹³; N¹⁴, N¹⁵, S³², S³⁴, O¹⁶, O¹⁷, and O¹⁸.

19. A method of comparatively analyzing protein-protein interactions between proteins present in two samples including a first sample and a second sample comprising the steps of:
(a) cross-linking the proteins in the first sample with the bifunctional cross-linking agent of claim 1,
(b) cross-linking proteins in the second sample with the bifunctional cross-linking agent of step (a);
(c) combining the first sample and second sample to produce a mixed sample; and
(d) analyzing the mixed sample.

20. The method of claim 19, further comprising the step of enriching the mixed sample before the analyzing step.

21. The method of claim 19, further comprising the step of enzymatically or chemically cleaving the bifunctional crosslinker present in the mixed sample before the analyzing step.

22. The method of claim 19, further comprising the step of capping the reactive groups produced by cleavage.

23. The method of claim 19, wherein the cleavage step comprises cleaving the proteins present in the mixed sample using a proteolytic agent.

24. The method of claim 19, wherein the bifunctional crosslinker, the target protein, or both bifunctional crosslinker, the target protein further comprise an affinity tag, and the enriching step comprises capturing the affinity tag in a chromatographic matrix.

25. The method of claim 19, wherein the analyzing step comprises comparing the amount protein fragment bound to the bifunctional cross-linked protein from the first sample and amount of protein fragment bound to the mass-shifting variant from the second sample.

26. The method of claim 19, wherein the first sample and the second sample are derived from different sources.

27. The method of claim 19, wherein the first sample and the second sample are derived from a single source, wherein the first sample comprises material that has been contacted with an effector agent and the second sample has not been contacted with the same effector agent.

28. The method of claim 19, where the first and second sample are derived from a mammalian subject before and after administering an effector agent to the mammalian subject.

29. The method of claim 20, wherein the enriching steps and the analyzing step occur in series without operator intervention.

30. The method of claim 19, wherein the analysis step comprises one or more MS technique selected from MALDI-TOF, ES, LC-ESI-MS, MALDI-TOF/TOF, ESI-MS-MS, FAB, FTICR-MS, and combinations thereof.

31. A differential isotopic labeling kit, comprising a symmetrical bifunctional crosslinker and an isotopic variant of the bifunctional crosslinker, wherein the symmetrical bifunctional crosslinker and the isotopic variant have a mass shift differential of at least 2 Da.

32. The kit of claim 31, wherein the symmetrical bifunctional crosslinker and the isotopic variant have mass shift differential of at least 4 Da.

33. The kit of claim 31, comprising a set of matching bifunctional cleavable crosslinkers, comprising a first bifunctional crosslinker and a second bifunctional crosslinker, a third bifunctional crosslinker, wherein each bifunctional crosslinker has a mass shift differential of at least 2 Da compared to the next lower mass bifunctional crosslinker.

34. The kit of claim 33, comprising a set of matching bifunctional cleavable crosslinkers, comprising a first bifunctional crosslinker and a second bifunctional crosslinker, a third bifunctional crosslinker such that all bifunctional crosslinkers have a mass shift differential of at least 4 Da compared to the next lower mass bifunctional crosslinker.

35. The kit of claim 31, wherein each of the matched bifunctional crosslinkers comprise first reactive terminal moiety including an amine reactive group and the second reactive terminal moiety comprises a reactive group selected from esters, aryl azides haloacyl, carboxyl, disulfides, maleimides, hydrazides, aldehydes, glyoxals, and imidoesters.
